(19) United States
(12) Patent Application Publication
(10) Pub. No.: US 2002/0102606 A1
(43) Pub. Date: Aug. 1, 2002

(54) Protein-protein interactions

(76) Inventors: Karen Heichman, Salt Lake City, UT (US); Paul L. Bartel, Salt Lake City, UT (US)

Correspondence Address: ROTHWELL, FIGG, ERNST & MANBECK, P.C., 555 13TH STREET, N.W., SUITE 701, EAST TOWER, WASHINGTON, DC, 20004, US

(21) Appl. No.: 09/847,599

(22) Filed: May 3, 2001

Related U.S. Application Data
(60) Provisional application No. 60/201,722, filed on May 4, 2000.

(51) Int. Cl. G01N 33/53; C12P 21/02; C12N 9/64; C12N 5/08

(52) U.S. Cl. 435/7.1; 435/226; 435/69.1; 435/368; 435/320.1

(57) ABSTRACT

The present invention relates to the discovery of novel protein-protein interactions that are involved in mammalian physiological pathways, including physiological disorders or diseases. Examples of physiological disorders and diseases include non-insulin dependent diabetes mellitus (NIDDM), neurodegenerative disorders, such as Alzheimer's Disease (AD), and the like. Thus, the present invention is directed to complexes of these proteins and/or their fragments, antibodies to the complexes, diagnosis of physiological generative disorders (including diagnosis of a predisposition to and diagnosis of the existence of the disorder), drug screening for agents which modulate the interaction of proteins described herein, and identification of additional proteins in the pathway common to the proteins described herein.

### CROSS-REFERENCE TO RELATED APPLICATION

The present application is related to U.S. provisional patent application Ser. No. 60/201,722 filed on May 4, 2000, incorporated herein by reference.

### BACKGROUND OF THE INVENTION

The present invention relates to the discovery of novel protein-protein interactions that are involved in mammalian physiological pathways, including physiological disorders or diseases. Examples of physiological disorders and diseases include non-insulin dependent diabetes mellitus (NIDDM), neurodegenerative disorders, such as Alzheimer's Disease (AD), and the like. Thus, the present invention is directed to complexes of these proteins and/or their fragments, antibodies to the complexes, diagnosis of physiological generative disorders (including diagnosis of a predisposition to and diagnosis of the existence of the disorder), drug screening for agents which modulate the interaction of proteins described herein, and identification of additional proteins in the pathway common to the proteins described herein.

The publications and other materials used herein to illuminate the background of the invention, and in particular, cases to provide additional details respecting the practice, are incorporated herein by reference, and for convenience, are referenced by author and date in the following text and respectively grouped in the appended Bibliography.

Many processes in biology, including transcription, translation and metabolic or signal transduction pathways, are mediated by non-covalently associated protein complexes. The formation of protein-protein complexes or protein-DNA complexes produce the most efficient chemical machinery. Much of modem biological research is concerned with identifying proteins involved in cellular processes, determining their functions, and how, when and where they interact with other proteins involved in specific pathways. Further, with rapid advances in genome sequencing, there is a need to define protein linkage maps, i.e., detailed inventories of protein interactions that make up functional assemblies of proteins or protein complexes or that make up physiological pathways.

Recent advances in human genomics research has led to rapid progress in the identification of novel genes. In applications to biological and pharmaceutical research, there is a need to determine functions of gene products. A first step in defining the function of a novel gene is to determine its interactions with other gene products in appropriate context. That is, since proteins make specific interactions with other proteins or other biopolymers as part of functional assemblies or physiological pathways, an appropriate way to examine function of a gene is to determine its physical relationship with other genes. Several systems exist for identifying protein interactions and hence relationships between genes.

There continues to be a need in the art for the discovery of additional protein-protein interactions involved in mammalian physiological pathways. There continues to be a need in the art also to identify the protein-protein interactions that are involved in mammalian physiological disorders and diseases, and to thus identify drug targets.

### SUMMARY OF THE INVENTION

The present invention relates to the discovery of protein-protein interactions that are involved in mammalian physiological pathways, including physiological disorders or diseases, and to the use of this discovery. The identification of the interacting proteins described herein provide new targets for the identification of useful pharmaceuticals, new targets for diagnostic tools in the identification of individuals at risk, sequences for production of transformed cell lines, cellular models and animal models, and new bases for therapeutic intervention in such physiological pathways

Thus, one aspect of the present invention is protein complexes. The protein complexes are a complex of (a) two interacting proteins, (b) a first interacting protein and a fragment of a second interacting protein, (c) a fragment of a first interacting protein and a second interacting protein, or (d) a fragment of a first interacting protein and a fragment of a second interacting protein. The fragments of the interacting proteins include those parts of the proteins, which interact to form a complex. This aspect of the invention includes the detection of protein interactions and the production of proteins by recombinant techniques. The latter embodiment also includes cloned sequences, vectors, transfected or transformed host cells and transgenic animals.

A second aspect of the present invention is an antibody that is immunoreactive with the above complex. The antibody may be a polyclonal antibody or a monoclonal antibody. While the antibody is immunoreactive with the complex, it is not immunoreactive with the component parts of the complex. That is, the antibody is not immunoreactive with a first interactive protein, a fragment of a first interacting protein, a second interacting protein or a fragment of a second interacting protein. Such antibodies can be used to detect the presence or absence of the protein complexes.

A third aspect of the present invention is a method for diagnosing a predisposition for physiological disorders or diseases in a human or other animal. The diagnosis of such disorders includes a diagnosis of a predisposition to the disorders and a diagnosis for the existence of the disorders. In accordance with this method, the ability of a first interacting protein or fragment thereof to form a complex with a second interacting protein or a fragment thereof is assayed, or the genes encoding interacting proteins are screened for mutations in interacting portions of the protein molecules. The inability of a first interacting protein or fragment thereof to form a complex, or the presence of mutations in a gene within the interacting domain, is indicative of a predisposition to, or existence of a disorder. In accordance with one embodiment of the invention, the ability to form a complex is assayed in a two-hybrid assay. In a first aspect of this embodiment, the ability to form a complex is assayed by a yeast two-hybrid assay. In a second aspect, the ability to form a complex is assayed by a mammalian two-hybrid assay. In a second embodiment, the ability to form a complex is assayed by measuring in vitro a complex formed by combining said first protein and said second protein. In one aspect the proteins are isolated from a human or other animal. In a third embodiment, the ability to form a complex is assayed by measuring the binding of an antibody, which is specific for the complex. In a fourth embodiment, the ability to form a complex is assayed by measuring the binding of an antibody that is specific for the complex with a tissue extract from a human or other animal. In a fifth embodiment, coding sequences of the interacting proteins described herein are screened for mutations.

A fourth aspect of the present invention is a method for screening for drug candidates which are capable of modulating the interaction of a first interacting protein and a second interacting protein. In this method, the amount of the complex formed in the presence of a drug is compared with the amount of the complex formed in the absence of the drug. If the amount of complex formed in the presence of the drug is greater than or less than the amount of complex formed in the absence of the drug, the drug is a candidate for modulating the interaction of the first and second interacting proteins. The drug promotes the interaction if the complex formed in the presence of the drug is greater and inhibits (or disrupts) the interaction if the complex formed in the presence of the drug is less. The drug may affect the interaction directly, i.e., by modulating the binding of the two proteins, or indirectly, e.g., by modulating the expression of one or both of the proteins.

A fifth aspect of the present invention is a model for such physiological pathways, disorders or diseases. The model may be a cellular model or an animal model, as further described herein. In accordance with one embodiment of the invention, an animal model is prepared by creating transgenic or “knock-out” animals. The knock-out may be a total knock-out, i.e., the desired gene is deleted, or a conditional knock-out, i.e., the gene is active until it is knocked out at a determined time. In a second embodiment, a cell line is derived from such animals for use as a model. In a third embodiment, an animal model is prepared in which the biological activity of a protein complex of the present invention has been altered. In one aspect, the biological activity is altered by disrupting the formation of the protein complex, such as by the binding of an antibody or small molecule to one of the proteins which prevents the formation of the protein complex. In a second aspect, the biological activity of a protein complex is altered by disrupting the action of the complex, such as by the binding of an antibody or small molecule to the protein complex which interferes with the action of the protein complex as described herein. In a fourth embodiment, a cell model is prepared by altering the genome of the cells in a cell line. In one aspect, the genome of the cells is modified to produce at least one protein complex described herein. In a second aspect, the genome of the cells is modified to eliminate at least one protein of the protein complexes described herein.

A sixth aspect of the present invention are nucleic acids coding for novel proteins discovered in accordance with the present invention and the corresponding proteins and antibodies.

A seventh aspect of the present invention is a method of screening for drug candidates useful for treating a physiological disorder. In this embodiment, drugs are screened on the basis of the association of a protein with a particular physiological disorder. This association is established in accordance with the present invention by identifying a relationship of the protein with a particular physiological disorder. The drugs are screened by comparing the activity of the protein in the presence and absence of the drug. If a difference in activity is found, then the drug is a drug candidate for the physiological disorder. The activity of the protein can be assayed in vitro or in vivo using conventional techniques, including transgenic animals and cell lines of the present invention.

### DETAILED DESCRIPTION OF THE INVENTION

The present invention is the discovery of novel interactions between proteins described herein. The genes coding for some of these proteins may have been cloned previously, but their potential interaction in a physiological pathway or with a particular protein was unknown. Alternatively, the genes coding for some of these proteins have not been cloned previously and represent novel genes. These proteins are identified using the yeast two-hybrid method and searching a human total brain library, as more fully described below.

According to the present invention, new protein-protein interactions have been discovered. The discovery of these interactions has identified several protein complexes for each protein-protein interaction. The protein complexes for these interactions are set forth below in Tables 1-41, which also identify the new protein-protein interactions of the present invention.

TABLE 1
Protein Complexes of MAPKAP-K2/HSP27 Interaction

MAP Kinase MAPKAP-K2 (MAPKAP-K2) and Heat Shock Protein
Hsp27 (HSP27)
A fragment of MAPKAP-K2 and HSP27
MAPKAP-K2 and a fragment of HSP27
A fragment of MAPKAP-K2 and a fragment of HSP27

TABLE 2
Protein Complexes of MAPKAP-X59131 Interaction

MAP Kinase MAPKAP-K3 (MAPKAP-K3) and Highly Charged Amino
Acid Sequence (X59131)
A fragment of MAPKAP-K3 and X59131
MAPKAP-K3 and a fragment of X59131
A fragment of MAPKAP-K3 and a fragment of X59131

TABLE 3
Protein Complexes of MAPKAP-K3/EZF Interaction

MAP Kinase MAPKAP-K3 (MAPKAP-K3) and EZF
A fragment of MAPKAP-K3 and EZF
MAPKAP-K3 and a fragment of EZF
A fragment of MAPKAP-K3 and a fragment of EZF

TABLE 4
Protein Complexes of MAPKAP-K3/KIAA0674 Interaction

MAP Kinase MAPKAP-K3 (MAPKAP-K3) and KIAA0674
A fragment of MAPKAP-K3 and KIAA0674
MAPKAP-K3 and a fragment of KIAA0674
A fragment of MAPKAP-K3 and a fragment of KIAA0674

TABLE 5
Protein Complexes of MAPKAP-K3/GM88 Interaction

MAP Kinase MAPKAP-K3 (MAPKAP-K3) and Golgi Apparatus
Protein 88 (GM88)
A fragment of MAPKAP-K3 and GM88
MAPKAP-K3 and a fragment of GM88
A fragment of MAPKAP-K3 and a fragment of GM88

TABLE 6
Protein Complexes of MAPKAP-K3/KIAA0216 Interaction

MAP Kinase MAPKAP-K3 (MAPKAP-K3) and KIAA0216
A fragment of MAPKAP-K3 and KIAA0216
MAPKAP-K3 and a fragment of KIAA0216
A fragment of MAPKAP-K3 and a fragment of KIAA0216

TABLE 7
Protein Complexes of MAPKAP-K3/RACK1 Interaction

MAP Kinase MAPKAP-K3 (MAPKAP-K3) and RACK1
A fragment of MAPKAP-K3 and RACK1
MAPKAP-K3 and a fragment of RACK1
A fragment of MAPKAP-K3 and a fragment of RACK1

TABLE 8
Protein Complexes of MAPKAP-K3/HRS Interaction

MAP Kinase MAPKAP-K3 (MAPKAP-K3) and Hepatocyte Growth
Factor-Regulated Tyrosine Kinase (HRS)
A fragment of MAPKAP-K3 and HRS
MAPKAP-K3 and a fragment of HRS
A fragment of MAPKAP-K3 and a fragment of HRS

TABLE 9
Protein Complexes of MAPKAP-K3/KRML Interaction

MAP Kinase MAPKAP-K3 (MAPKAP-K3) and KRML
A fragment of MAPKAP-K3 and KRML
MAPKAP-K3 and a fragment of KRML
A fragment of MAPKAP-K3 and a fragment of KRML

TABLE 10
Protein Complexes of MAPKAP-K3/TOM1 Interaction

MAP Kinase MAPKAP-K3 (MAPKAP-K3) and TOM1
A fragment of MAPKAP-K3 and TOM1
MAPKAP-K3 and a fragment of TOM1
A fragment of MAPKAP-K3 and a fragment of TOM1

TABLE 11
Protein Complexes of MAPKALP-K3/TMP3 Interaction

MAP Kinase MAPKAP-K3 (MAPKAP-K3) and Tropomyosin 3 (TMP3)
A fragment of MAPKAP-K3 and TMP3
MAPKAP-K3 and a fragment of TMP3
A fragment of MAPKAP-K3 and a fragment of TMP3

TABLE 12
Protein Complexes of MAPKAP-K3/ZFM1 Interaction

MAP Kinase MAPKAP-K3 (MAPKAP-K3) and ZFM1
A fragment of MAPKAP-K3 and ZFM1
MAPKAP-K3 and a fragment of ZFM1
A fragment of MAPKAP-K3 and a fragment of ZFM1

TABLE 13
Protein Complexes of MAPKAP-K3/Homer-3 Interaction

MAP Kinase MAPKAP-K3 (MAPKAP-K3) and Homer-3
A fragment of MAPKAP-K3 and Homer-3
MAPKAP-K3 and a fragment of Homer-3
A fragment of MAPKAP-K3 and a fragment of Homer-3

TABLE 14
Protein Complexes of MAPKAP-K3/MAX Interaction

MAP Kinase MAPKAP-K3 (MAPKAP-K3) and MAX
A fragment of MAPKAP-K3 and MAX
MAPKAP-K3 and a fragment of MAX
A fragment of MAPKAP-K3 and a fragment of MAX

TABLE 15
Protein Complexes of MAPKAP-K3/ERF-2 Interaction

MAP Kinase MAPKAP-K3 (MAPKAP-K3) and ERF-2
A fragment of MAPKAP-K3 and ERF-2
MAPKAP-K3 and a fragment of ERF-2
A fragment of MAPKAP-K3 and a fragment of ERF-2

TABLE 16
Protein Complexes of MAPKAP-K3/Vimentin Interaction

MAP Kinase MAPKAP-K3 (MAPKAP-K3) and Vimentin
A fragment of MAPKAP-K3 and Vimentin
MAPKAP-K3 and a fragment of Vimentin
A fragment of MAPKAP-K3 and a fragment of Vimentin

TABLE 17
Protein Complexes of MAPKAP-K3/NuMA1 Interaction

MAP Kinase MAPKAP-K3 (MAPKAP-K3) and Nuclear Mitotic
Apparatus Protein 1 (NuMA1)
A fragment of MAPKAP-K3 and NuMA1
MAPKAP-K3 and a fragment of NuMA1
A fragment of MAPKAP-K3 and a fragment of NuMA1

TABLE 18
Protein Complexes of MAPKAP-K3/HSPC161 Interaction

MAP Kinase MAPKAP-K3 (MAPKAP-K3) and HSPC161
A fragment of MAPKAP-K3 and HSPC161
MAPKAP-K3 and a fragment of HSPC161
A fragment of MAPKAP-K3 and a fragment of HSPC161

TABLE 19
Protein Complexes of MAPKAP-K3/KIAA1026 Interaction

MAP Kinase MAPKAP-K3 (MAPKAP-K3) and KIAA1026
A fragment of MAPKAP-K3 and KIAA1026
MAPKAP-K3 and a fragment of KIAA1026
A fragment of MAPKAP-K3 and a fragment of KIAA1026

TABLE 20
Protein Complexes of MAPKAP-K3/HSP27 Interaction

MAP Kinase MAPKAP-K3 (MAPKAP-K3) and Heat Shock
Protein 27 (HSP27)
A fragment of MAPKAP-K3 and HSP27
MAPKAP-K3 and a fragment of HSP27
A fragment of MAPKAP-K3 and a fragment of HSP27

TABLE 21
Protein Complexes of L130/Dynactin Interaction

Leucine Rich Protein L130 (L130) and Dynactin
A fragment of L130 and Dynactin
L130 and a fragment of Dynactin
A fragment of L130 and a fragment of Dynactin

TABLE 22
Protein Complexes of L130/CREBL2 Interaction

Leucine Rich Protein L130 (L130) and CRE-Binding Protein-Like 2
(CREBL2)
A fragment of L130 and CREBL2
L130 and a fragment of CREBL2
A fragment of L130 and a fragment of CREBL2

TABLE 23
Protein Complexes of PRAK/MLK2 Interaction

Protein Kinase PRAK (PRAK) and MLK2
A fragment of PRAK and MLK2
PRAK and a fragment of MLK2
A fragment of PRAK and a fragment of MLK2

TABLE 24
Protein Complexes of PRAK/Tenascin XB Interaction

Protein Kinase PRAK (PRAK) and Tenascin XB
A fragment of PRAK and Tenascin XB
PRAK and a fragment of Tenascin XB
A fragment of PRAK and a fragment of Tenascin XB

TABLE 25
Protein Complexes of PRAK/Golgin-95 Interaction

Protein Kinase PRAK (PRAK) and Golgin-95
A fragment of PRAK and Golgin-95
PRAK and a fragment of Golgin-95
A fragment of PRAK and a fragment of Golgin-95

TABLE 26
Protein Complexes of PRAK/Kendrin Interaction

Protein Kinase PRAK (PRAK) and Kendrin
A fragment of PRAK and Kendrin
PRAK and a fragment of Kendrin
A fragment of PRAK and a fragment of Kendrin

TABLE 27
Protein Complexes of PRAK/KIAA0555 Interaction

Protein Kinase PRAK (PRAK) and KIAA0555
A fragment of PRAK and KIAA0555
PRAK and a fragment of KIAA0555
A fragment of PRAK and a fragment of KIAA0555

TABLE 28
Protein Complexes of PRAK/NuMA1 Interaction

Protein Kinase PRAK (PRAK) and and Nuclear Mitotic Apparatus
Protein 1 (NuMA1)
A fragment of PRAK and NuMA1
PRAK and a fragment of NuMA1
A fragment of PRAK and a fragment of NuMA1

TABLE 29
Protein Complexes of PRAK/ABP620 Interaction

Protein Kinase PRAK (PRAK) and ABP620
A fragment of PRAK and ABP620
PRAK and a fragment of ABP620
A fragment of PRAK and a fragment of ABP620

TABLE 30
Protein Complexes of PRAK/Dynactin Interaction

Protein Kinase PRAK (PRAK) and Dynactin
A fragment of PRAK and Dynactin
PRAK and a fragment of Dynactin
A fragment of PRAK and a fragment of Dynactin

TABLE 31
Protein Complexes of PRAK/SMN1 Interaction

Protein Kinase PRAK (PRAK) and Survival Motor Neuron 1 (SMN1)
A fragment of PRAK and SMN1
PRAK and a fragment of SMN1
A fragment of PRAK and a fragment of SMN1

TABLE 32
Protein Complexes of PRAK/HAT1 Interaction

Protein Kinase PRAK (PRAK) and Histone Acetyl Transferase 1 (HAT1)
A fragment of PRAK and HAT1
PRAK and a fragment of HAT1
A fragment of PRAK and a fragment of HAT1

TABLE 33
Protein Complexes of PRAK/Homer-3 Interaction

Protein Kinase PRAK (PRAK) and Homer-3
A fragment of PRAK and Homer-3
PRAK and a fragment of Homer-3
A fragment of PRAK and a fragment of Homer-3

TABLE 34

Protein Complexes of PRAK/Kinectin Interaction
Protein Kinase PRAK (PRAK) and Kinectin
A fragment of PRAK and Kinectin
PRAK and a fragment of Kinectin
A fragment of PRAK and a fragment of Kinectin

TABLE 35
Protein Complexes of PRAK/Bicaudal-D Interaction

Protein Kinase PRAK (PRAK) and Bicaudal-D
A fragment of PRAK and Bicaudal-D
PRAK and a fragment of Bicaudal-D
A fragment of PRAK and a fragment of Bicaudal-D

TABLE 36
Protein Complexes of TIAR/Profilin II Interaction

TIAR and Profilin II
A fragment of TIAR and Profilin II
TIAR and a fragment of Profilin II
A fragment of TIAR and a fragment of Profilin II

TABLE 37
Protein Complexes of TIAR/SEI1 Interaction

TIAR and SEI1
A fragment of TIAR and SEI1
TIAR and a fragment of SEI1
A fragment of TIAR and a fragment of SEI1

TABLE 38
Protein Complexes of p38 alpha/WPB-2 Interaction

Protein Kinase p38 alpha (p38 alpha) and WPB-2
A fragment of p38 alpha and WPB-2
p38 alpha and a fragment of WPB-2
A fragment of p38 alpha and a fragment of WPB-2

TABLE 39
Protein Complexes of p38 alpha/JNK2 Interaction

Protein Kinase p38 alpha (p38 alpha) and JNK2
A fragment of p38 alpha and JNK2
p38 alpha and a fragment of JNK2
A fragment of p38 alpha and a fragment of JNK2

TABLE 40
Protein Complexes of p38 gamma/DLG-2 Interaction

Protein Kinase p38 gamma (p38 gamma) and DLG-2
A fragment of p38 gamma and DLG-2
p38 gamma and a fragment of DLG-2
A fragment of p38 gamma and a fragment of DLG-2

TABLE 41
Protein Complexes of C-NAP-1/MYT1 Interaction

C-NCAP-1 and Myelin Transcription Factor 1 (MYT1)
A fragment of C-NCAP-1 and MYT1
C-NCAP-1 and a fragment of MYT1
A fragment of C-NCAP-1 and a fragment of MYT1

The involvement of above interactions in particular pathways is as follows.

Many cellular proteins exert their function by interacting with other proteins in the cell. Examples of this are found in the formation of multiprotein complexes and the association of an enzymes with their substrates. It is widely believed that a great deal of information can be gained by understanding individual protein-protein interactions, and that this is useful in identifying complex networks of interacting proteins that participate in the workings of normal cellular functions. Ultimately, the knowledge gained by characterizing these networks can lead to valuable insight into the causes of human diseases and can eventually lead to the development of therapeutic strategies. The yeast two-hybrid assay is a powerful tool for determining protein-protein interactions and it has been successfully used for studying human disease pathways. In one variation of this technique, a protein of interest (or a portion of that protein) is expressed in a population of yeast cells that collectively contain all protein sequences. Yeast cells that possess protein sequences that interact with the protein of interest are then genetically selected, and the identity of those interacting proteins are determined by DNA sequencing. Thus, proteins that can be demonstrated to interact with a protein known to be involved in a human disease are therefore also implicated in that disease. To create a more complex network of interactions in a disease pathway, proteins which were identified in the first round of two-hybrid screening are subsequently used in two-hybrid assays as the protein of interest.

Cellular events that are initiated by exposure to growth factors, cytokines and stress are propagated from the outside of the cell to the nucleus by means of several protein kinase signal transduction cascades. p38 kinase is a member of the MAP kinase family of protein kinases. It is a key player in signal transduction pathways induced by the proinflammatory cytokines such as tumor necrosis factor (TNF), interleukin-1 (IL-1) and interleukin-6 (IL-6) and it also plays a critical role in the synthesis and release of the proinflammatory cytokines (Raingeaud et al., 1995; Lee et al., 1996; Miyazawa et al., 1998; Lee et al., 1994). Studies of inhibitors of p38 kinase have shown that blocking p38 kinase activity can cause anti-inflammatory effects, thus suggesting that this may be a mechanism of treating certain inflammatory diseases such as rheumatoid arthritis and inflammatory bowel disease. Further, p38 kinase activity has been implicated in other human diseases such as atherosclerosis, cardiac hypertrophy and hypoxic brain injury (Grammer et al., 1998; Mach et al., 1998; Wang et al., 1998; Nemoto et al., 1998; Kawasaki et al., 1997). Thus, by understanding p38 function, one may gain novel insight into a cellular response mechanism that affects a number of tissues and can potentially lead to harmful affects when disrupted.

The search for the physiological substrates of p38 kinase has taken a number of approaches including a variety of biochemical and cell biological methods. There are four known human isoforms of p38 kinase termed alpha, beta, gamma and delta, and these are thought to possess different physiological functions, likely because they have distinct substrate and tissue specificities. Some of the p38 kinase substrates are known, and the list includes transcription factors and additional protein kinases that act downstream of p38 kinase. Four of the kinases that act downstream of p38 kinase, MAPKAP-K2, MAPKAP-K3, PRAK and MSK1, are currently being analyzed themselves and some of their substrates have been identified.

The yeast two-hybrid system has been used to detect potential substrates and upstream regulators of the p38 kinases and their downstream kinases. In a two-hybrid search using p38 alpha kinase as the protein of interest, three proteins were shown to bind to p38 alpha. The first, protein found to bind to p38 alpha kinase is WBP-2. WBP-2 was originally identified as a putative ligand of the WW domain of YAP (Yes-associated protein) (Chen et al., 1997). The WW domains have been implicated in mediating protein-protein interactions, and they appear to bind specifically to a so-called PY motif, a proline-rich domain followed by a tyrosine residue. WBP-2 has several such PY motifs in its C-terminus although its function is not currently well characterized. Interestingly, several additional WW domain proteins have been identified by virtue of their ability to bind to PY and PY-like motifs, and these include signaling or regulatory proteins such as RasGAP, AP-2, p53, and IL-6 receptor alpha (Pirozzi et al., 1997). Thus, the finding that p38 alpha can bind to WBP-2 suggests that p38 alpha might be capable of influencing the function of important signaling and regulatory proteins via its interaction with a protein, WBP-2. Further evidence linking WBP-2 to inflammation has been found using the two-hybrid system: Myriad Genetics has shown that WBP-2 also binds to TAB2, or TAK1 binding protein. TAB2 ihas been shown to be involved in IL-1 signalling (Takaesu et al., 2000). The second protein, JNK2, is itself a member of the MAP kinase family like the p38 kinases. JNK2 phosphorylates transcription factors and it becomes activated itself by phosphorylation (Sluss et al., 1994). JNK2 is activated following exposure to TNF alpha, and it is generally held that it plays a key role in the inflammatory response. Interestingly, the JNK2 protein sequence contains at least 2 consensus phosphorylation sites for the p38 kinases, thus it is very likely that JNK2 may act as a substrate for p38 alpha.

In a two-hybrid search using p38 gamma kinase as the protein of interest, a single interactor, DLG2 (or chapsyn-110), has been shown to be an interactor. DLG2 is a channel-associated that belongs to the MAGUK (membrane-associated guanylate kinase) family of cell junction proteins. It possesses a number of important structural features including an SH3 domain, a guanylate kinase-like domain and three PDZ domains. DLG2 has been shown to interact with the cytoplasmic tail of NMDA receptor subunits and potassium channels (Kim et al., 1996). The interaction between DLG2 and p38 gamma seems very plausible since the extreme C-terminus of p38 gamma contains a consensus PDZ-binding motif, and the region of DLG2 that associates with p38 gamma in the two-hybrid assay (amino acids 294 to 594) contains a PDZ domain. Furthermore, DLG2 may be capable of acting as a substrate for p38 gamma since the sequence of DLG2 contains a number of MAP kinase consensus phosphorylation sites. However, none of the other p38 kinases possess a PDZ-binding motif at its C-terminus, therefore suggesting that the interaction between DLG2 and p38 gamma is specific.

MAPKAP-K2, a protein kinase that acts downstream of p38 kinase in the same signal transduction pathway, was used in a two-hybrid search to identify potential substrates or regulators. MAPKAP-K2 was demonstrated to interact with the heat shock protein Hsp27. When overexpressed, Hsp27 can protect cells from heat shock and oxidative stress (Rogalla et al., 1999). Small heat shock proteins such as Hsp27 have been demonstrated to become phosphorylated by MAPKAP kinases in response to extracellular stresses, therefore it is not surprising that we could detect the interaction between Hsp27 and MAPKAP-K2. Interestingly, this protein was also identified as a two-hybrid interactor of another highly related p38-activated protein kinase termed MAPKAP-K3. In fact, the regions of MAPKAP-K2 and MAPKAP-K3 that interacted with Hsp27 both correspond to the C-terminal portions of the kinase domains. Furthermore, MAPKAP-K2 and MAPKAP-K3 interacted with the same region of Hsp27.

When a second p38-activated protein kinase, MAPKAP-K3, was used in a two-hybrid search 19 proteins were demonstrated to bind to it. Several structural proteins are included in the list of MAPKAP-K3 interactors. The first structural protein to bind to MAPKAP-K3, tropomyosin 3, is a structural protein and it also plays a role in muscle contraction when it is expressed in muscle cells. Its role in non-muscle cells is unclear, but there have been several reports that the related tropomyosin 1 plays a role in preventing the unregulated cell growth characteristic of transformed or cancer cells (Prasad et al., 1993). The second structural MAPKAP-K3 binding-protein detected in the yeast two-hybrid assay is the cytoskeletal intermdiate filament protein vimentin. Vimentin is a coiled-coil protein that exists as a phosphoprotein; interestingly, its phosphorylation state is increased during cell division when filaments are being reorganized (Evans, 1988). There appears to be at least a few sites within the vimentin protein sequence that may act as MAPKAP-K3 phophorylation sites.

Finally, MAPKAP-K3 has been shown to interact with the NuMA1 nuclear mitotic apparatus protein in the yeast two-hybrid assay. NuMA1 was previously shown by us to interact with the Akt1 and Akt2 protein kinases that have been implicated in the signal transduction pathway involved in the inflammatory response. NuMA1 is a very interesting protein since it shuttles between the nucleus and the cytoplasm in a cell cycle-dependent manner (Lydersen and Pettijohn, 1980). Furthermore, NuMA1 is phosphorylated in a cell cycle-regulated fashion which seems to be critical for its function (Sparks wt al., 1995). The amino acid sequence of NuMA1 suggests that it contains a calponin-homology region at its N-terminus and spectrin repeats at the C-terminal end. The C-terminal end also appears to contain 5 MAPKAP consensus phosphorylation sites. It is interesting to note that MAPKAP-K3 and the Akts bind to NuMA1 in distinct regions; MAPKAP-K3 binds to amino acids 413 to 519, and the Akts bind to a region including residues 98 to 365. Interestingly, NuMA1 has been shown to interact with another kinase related to MAPKAP-K3 called PRAK. PRAK and MAPKAP-K3 interact with the same region of NuMA1, again adding credence to the idea that NuMA1 is a substrate of the MAPKAP kinases.

MAPKAP-K3 has also been demonstrated to interact with several proteins that appear to be functional in transcriptional regulation or post-translational regulation of RNAs. The first such protein is a putative transcription factor, EZF, that is similar to the Krueppel protein of Drosophila melanogaster. EZF (also known as GKLF in mice) has three C2H2-type zinc fingers that reside in the C-terminus of the protein. This C-terminal region corresponds to the segment that binds to the kinase domain of MAPKAP-K3. Human EZF was cloned from vascular endothelial cells although studies in mice have shown that it is expressed in several other tissues (Shields, et al., 1996). It has been suggested that EZF is a negative regulator of cell growth.

The second protein, MAX, is a well-characterized basic helix-loop-helix leucine zipper-containing transcription factor that was originally identified since it heterodimerized with the proto-oncogene c-myc (Blackwood and Eisenman, 1991). It has been demonstrated that in this capacity, MAX acts as a transcriptional activator. In constrast, MAX has also been shown to heterodimerize with the MAD protein (and other similar proteins) to form a transcriptional repressor (Ayer et al., 1993). It is thought that MAD plays a pivotal role in gene regulatory mechanisms since its choice of binding partner can determine whether important cell cycle and developmental genes are transcriptionally activated or silenced.

The same region of MAPKAP-K3 has also been shown to bind to the ERF-2 transcriptional regulator (also known as TIS11D). ERF-2 (EGF response factor) is a zinc finger containing nuclear protein that was originally identifed in mouse as the TIS11D protein (Varnum et al., 1991). Interestingly, ERF-2 is similar to ERF-1 (or TISB), another EGF response factor, as well as the TTP protein that functions in regulating TNF-alpha transcript (Carballo et al., 1998). Further, the region of ERF-2 that is most similar to TTP (amino acids 152 to 203) is roughly the same as that which binds to MAPKAP-K3. Finally, inspection of the sequence of ERF-2 reveals 3 potential MAPKAP phosphorylation sites located throughout the protein. This exciting result suggests that MAPKAP-K3 may play a direct role in TNF-alpha regulation.

MAPKAP-K3 has been shown to bind to the transcriptional regulator KRML (also known as MafB) in a two-hybrid search. KRML is a basic leucine zipper-containing protein similar to AP-1 that binds to the Ets-1 transcriptional activator and represses transcription (Sieweke et al., 1996). KRML expression seems to be restricted to myelomonocytic cells. It appears to inhibit erythroid differentiation since its overexpression in an erythroblast cell line prevents differentiation without affecting cell proliferation.

Using a bait consisting of the C-terminal two-thirds of MAPKAP-K3 in a two-hybrid search, the transcriptional repressor ZFM1 was shown to be an interactor. ZFM1 is identical to the presplicing factor SF1, and its gene is located at the locus linked to multiple endocrine neoplasia type 1 (MEN1). ZFM1 has been shown by others to interact with the transcription activation domain of the sea urchin stage-specific activator protein (SSAP) (Zhang and Childs, 1998); furthermore, it has also been shown to bind to the SSAP-like human protein EWS protein and other proteins similar to EWS (Zhang et al., 1998). EWS functions in transcriptional regulation, and it is involved in cellular transformation events associated with Ewing's sarcoma. Myriad Genetics has shown that ZFM1 interacts with other proteins in the two-hybrid assay that are linked to transcription regulation and mRNA processing: LSF and U2AF2. The finding that MAPKAP-K3 can interact with ZFM1 suggests that MAPKAP-K3 may be capable of utilizing ZFM1 as a substrate, and this notion is further supported by the fact that ZFM1 contains a putative MAPKAP phosphorylation site.

In a two-hybrid search using the C-terminal two-thirds of the kinase domain of MAPKAP-K3 (amino acids 114 to 304), the double zinc finger protein HRS (hepatocyte growth factor-regulated tyrosine kinase) was shown to be an interactor. HRS is a phosphotyrosine protein and its tyrosine phosphorylation is induced upon stimulation with IL-2 (Asao et al., 1997), and it has been shown to bind to STAM (signal-transducing adaptor molecule), another protein that is tyrosine phosphorylated following IL-2 exposure. These two proteins, HRS and STAM, are involved in cytokine-mediated cell growth signaling and interact with one another via coiled-coil domains. Myriad Genetics has shown that in the two-hybrid assay HRS interacts with another protein linked to inflammation, the socalled NMI protein. NMI interacts with TAK1, or TGF-beta activated kinase 1 a. Along these lines, it is interesting to note that TAK1 interacts with TAB2, a protein shown to interacts with WBP-2, a p38 alpha kinase interactor. MAPKAP-K3 has also been shown to bind to TOM1, a protein similar to both HRS and STAM, therefore MAPKAP-K3 may be recognizing a common structural element in these related proteins. TOM1 is the human homolog of the chicken TOM1 gene that was shown to be transcriptionally regulated by myb (Burk and Klempnauer, 1999). All three contain coiled-coil domains that at least partially coincide with the regions that interact with MAPKAP-K3, however the areas of the proteins that are most alike lie outside of the coiled-coil region.

MAPKAP-K3 has also been shown to interact with 2 proteins that are involved in various aspects of signal transduction. The first protein, RACK1, is a WD repeat-containing protein that acts as an anchor for protein kinase C and is involved in its translocation and activation. RACK1 appears to play a role in TNF alpha release since it has been recently reported that a decrease in the level of RACK1 correlated with an age-associated change in the release of TNF alpha from LPS-stimulated macrophages (Corsini et al., 1999). This is an exciting finding since the interaction of MAPKAP-K3 and RACK1 suggests there may be a link between MAPKAP-K3 and TNF alpha release. RACK1 has a single MAPKAP consensus phosphorylation site, and it is tempting to speculate that RACK1's function might be regulated by MAPKAP-K3 phosphorylation.

MAPKAP-K3 has also been demonstrated to interact with the Homer-3 synaptic protein in a two-hybrid search of the spleen library. The Homer proteins have been shown by Tu et al. (1998) to physically link metabotropic glutamate receptors with the inositol triphosphate receptors, a very important process involved in intracellular calcium release. Although most of what is known about the Homer proteins has been discovered in neuronal systems, our findings point to additional functions in other cell types since Homer-3 was found in a two-hybrid search of the spleen library. Interestingly, the Homer-3 protein contains three MAPKAP consensus phosphorylation sites suggesting that its function may be modulated by MAPKAP-K3 phosphorylation. Myriad Genetics has shown that Homer-3 interacts with the related inflammation associated kinases MNK1 and PRAK. The region of Homer-3 that binds to all three kinases is included on a fragment containing residues 228 to 354.

MAPKAP-K3 has been shown to interact with a protein, GM88 or golgin-67, that does not necessarily fit into any specific functional categories with other MAPKAP-K3 interactors. GM-88 is an 88 kilodalton protein of the Golgi apparatus that does not have any known function though it does have 2 predicted MAPKAP consensus phosphorylation sites. There also do not appear to be any recognizable functional domains that lend insight into its function. One interesting comment, however, is that golgin-88 bears some protein sequence similarity to golgin-95, another Golgi protein that has been shown to interact with the related MAPKAP kinase, PRAK (see below).

Finally, MAPKAP-K3 has been found to interact with several proteins of unknown function. The first protein of unknown function, a highly charged amino acid sequence entered into GenBank under the accession X59131, is predicted to be 1092 amino acids in length. It contains a predicted ubiquitin protease motif between residues 227 to 501 and has 4 putative MAPKAP phosphorylation sites, therefore indicating that it may be a true substrate for MAPKAP-K3. The second protein of unknown function demonstrated to interact with MAPKAP-K3 in the yeast two-hybrid assay is known as KIAA0674. There appears to be an FKBD-type peptidyl-prolyl cis-trans isomerase domain in the first half of the known KIAA0674 protein sequence (amino acids 212 to 305) that sheds some light on the function of this putative protein. The segment of KIAA0674 (residues 891 to 1214) that interacts with MAPKAP-K3, however, lies distal to this predicted FKBD domain. A survey of ESTs (expressed sequence tags) matching the KIAA0674 nucleotide sequence indicates that it is expressed in a wide variety of tissues. In addition, there exist several MAPKAP consensus phosphorylation sites throughout the protein fragment, lending credence to the idea that KIAA0674 may act as a substrate for MAPKAP-K3.

The third protein of unknown function to interact with MAPKAP-K3 is the hypothetical KIAA0216 protein. The predicted protein sequence of this gene encodes a large protein of 1581 amino acids, a bipartite nuclear localization sequence, a RecA-homology region and several spectrin repeats. Strikingly, KIAA0216 contains 7 consensus MAPKAP phosphorylation sites, thereby strengthening the hypothesis that this protein is a substrate of MAPKAP-K3. Additionally, MAPKAP-K3 has been shown to interact with KIAA1026, a hypothetical protein of unknown function. There do not appear to be any obvious structural elements that lend much insight into this protein other than several putative nuclear localization sequences and 3 consensus MAPKAP phosphorylation sites Lastly, MAPKAP-K3 has been demonstrated to interact with a protein of unknown function called HSPC161. HSPC161 is a relatively small protein that has no known function nor does it have any recognizable functional domains that lend insight into it. Inspection of homologous ESTs indicate that HSPC161 is expressed in a wide variety of tissues. There do not appear to be an exact matches to the MAPKAP consensus phosphorylation site present in HSPC161, however there is one similar site in the N-terminus of the protein.

All 19 of these proteins described above may act as substrates for the MAPKAP-K3 protein kinase since they have been demonstrate to bind to it. It is possible that as a result of an extracellular stimulus and the ensuing signal transduction pathway that is transmitted by p38 and MAPKAP-K3 protein kinases, these cellular proteins are phosphorylated, thereby altering their cellular functions.

A third p38-activated protein kinase similar to MAPKAP-K2 and MAPKAP-K3, PRAK, was used in a two-hybrid assay and it was found to bind to 11 proteins. The first of these proteins, the MLK2 mixed lineage kinase, is involved in signal transduction like PRAK. MLK2 is a serine/threonine kinase with similarity to the MAP3K family of kinases. It contains some interesting structural domains in addition to its kinase domain, and these include an SH3 domain, a leucine zipper, and a CRIB (Rac/Cdc42 GTPase-binding) motif. MLK2 has been demonstrated to interact with several other proteins in two-hybrid experiments reported in the literature, and these include interactions with Rac, Cdc42, 14-3-3 eta, the kinesin motor protein KIF3X and the KIF3 targeting protein KAP3A (Nagata et al., 1998). Further, these same authors have shown that transfection of MLK2 into COS cells leads to the activation JNK, ERK and p38 kinase cascades, and they therefore conclude that there may be a link between stress activation and motor protein function. The finding that PRAK and MLK2 associate strengthens this argument. The second protein shown to bind to PRAK is the extracellular matrix protein tenascin XB. No functions have yet been ascribed to the tenascins, however a recent finding suggests that tenascin XB may be involved in connective tissue disorders akin to Ehlers-Danlos syndrome (Burch et al., 1997). Tenascin XB contains several consensus MAPKAP phosphorylation sites, so it may be capable of being recognized by PRAK in the context of a yeast two-hybrid setting.

The third protein found to interact with PRAK is the golgin-95 protein. Golgin-95 is a protein of the Golgi apparatus that is a human autoantigen (Fritzler et al., 1993). Its function is largely unknown but it is suspected to play a role in vesicular transport of proteins. PRAK can also bind to a centrosome component called kendrin or pericentrin. Kendrin is an extremely large protein of 3320 amino acids that does not appear to be well-characterized, however recent studies suggest that kendrin may function by anchoring cAMP-dependent protein kinase to the centrosomes (Diviani et al., 2000). It is very likely that kendrin can act as a substrate for PRAK since there appear to be 8 MAPKAP consensus sequences within the kendrin protein sequence.

PRAK has also been shown to interact with another cytoskeletal protein called dynactin. Dynactin is involved in the movement of vesicles and organelles along microtubules, and it contains a serine-rich region towards its amino-terminus (amino acids 156 to 183) and several coiled-coil regions throughout the remainder of the protein (Karki and Holzbaur, 1999). In a related finding, PRAK has been shown to associate with the kinesin motor anchoring protein, kinectin, in two-hybrid assays and kinectin has been previously been shown by us to interact with MAPKAP-K3. Kinectin is a very large coiled-coil containing integral membrane protein of the endoplasmic reticulum, however it does have a predicted bipartite nuclear localization sequence (Sheetz, 1999). Since kinectin interacts with the kinase domains of PRAK and MAPKAP-K3, it is tempting to speculate that the regulation of microtubule-dependent transport may be affected by phosphorylation. In support of this notion, kinectin appears to have 2 consensus phosphorylation sites for the MAPKAP family of kinases.

PRAK has also been shown to interact with the bicaudal-D protein in the yeast two-hybrid assay. The human form of bicaudal-D is a coiled-coil containing cytoskeletal protein whose function has been well studied in Drosophila. The fruitfly bicaudal-D protein has been shown to form a complex with egalitarian protein, and this complex has been demonstrated to be important for oocyte differentiation and patterning (Mach and Lehmann, 1997). Specifically, the egalitarian-bicaudal-D complex is needed to transport differentiation-promoting factors during early oogenesis, and in later stages, it is required for sorting of RNA molecules required for patterning in the embryo. Perhaps in human cells, bicaudal-D can act in a similar fashion and bind to critical RNA molecules thereby affecting their translation. The human bicaudal-D protein sequence contains several MAPKAP consensus phosphorylation sites. For this reason, it is tempting to speculate that PRAK may be capable of phosphorylating bicaudal-D and consequently influencing its function.

PRAK has been shown to be involved with another protein linked to RNA, SMN1. SMN1 (survival motor neuron protein 1) is a nuclear protein with three proline-rich regions toward the carboxy-terminus of the protein. It has been implicated in mRNA processing and is thought to play a key role in the biogenesis of small nuclear ribonucleoprotein particles (snRNPS) (Lorson et al., 1999). The SMN1 gene has been connected to the common genetic disease spinal muscular atrophy, an eventually lethal disorder characterized by muscle weakness and atrophy. SMN1 is expressed in a variety of tissues including brain, kidney, liver, cardiac and skeletal muscle, fibroblasts and lymphocytes, therefore it may participate in additional processes unrelated to muscle development or maintenance. The finding that PRAK interacts with SMN1 is an intriguing one since it links PRAK to RNA processing. PRAK has been shown to bind to a protein intimately linked to chromatin structure, the HAT1 histone acetyl transferase. HAT1 is a nuclear protein during the S phase of the cell cycle (Verreault et al., 1998). PRAK interacts with the extreme C-terminus of the HAT1 protein (amino acids 334 to 419) which incidentally contains a MAPKAP consensus phosphorylation site, thereby indicating that HAT1 may be a substrate of PRAK.

Finally, PRAK has been demonstrated to interact with two proteins of unknown function. The first of these, KIAA0555, is a hypothetical protein with no recognizable functional domains other than a region similar to the S. pombe cdc15 protein which is involved in actin re-organization (Fankhauser et al., 1995). KIAA0555 does contain 3 putative MAPKAP consensus phosphorylation sites. The second protein of unknown function to bind to PRAK in a yeast two-hybrid assay is ABP620. ABP620 is a very large protein of 5430 amino acids that is relatively uncharacterized except for the finding that it binds to actin. The N-terminal portion of the ABP620 contains a calponin-homology domain and virtually the rest of the protein is composed of spectrin repeats (35 total). ABP620 also appears to contain a bipartite nuclear localization sequence and several MAPKAP consensus phosphorylation sites, suggesting that it may act as a substrate for PRAK in the nucleus. This finding is particularly exciting since it serves to link PRAK to the actin cytoskeleton. All of these proteins may act as substrates for PRAK and their functions may be altered by phophorylation. Their association with PRAK links them to the inflammatory response and to the diseases that are related to this response.

Yeast two-hybrid searches have been performed using the KIAA0555 protein that was identified as an interactor of PRAK. In one search, the 14-3-3 epsilon signal transduction protein was identified as an interactor of KIAA0555. The 14-3-3 family of proteins are known to bind to phosphoserine residues (Takaesu et al., 2000). This finding is interesting since if KIAA0555 binds to 14-3-3 epsilon, it is suggestive that KIAA0555 is a phosphoserine-containing protein. Since KIAA0555 also binds to PRAK, a serine-specific protein kinase, and KIAA0555 has 3 putative MAPKAP family phosphorylation sites, it further strengthens the argument that KIAA0555 is a true substrate of PRAK.

Yeast two-hybrid searches have been performed using a leucine-rich protein of unknown function called L130 that was previously identified by us to be a common interactor of both MAPKAP-K2 and PRAK. L130 was originally identified by virtue of its high level of expression in hepatoblastoma cells (Hou et al., 1994), however there is currently no information about its function. Its expression in hepatoblastoma cells suggests a role in liver function or in the transformation of normal cells to malignant ones. L130 has been shown to interact with 2 proteins in two-hybrid searches. An intriguing finding is that L130 associates with dynactin in the two-hybrid. Recall that dynactin was also identified as an interactor of PRAK and that it is a cytoskeletal protein involved in the movement of vesicles and organelles along microtubules. L130 has been shown to bind to the potential transcription factor CRE-binding protein-like 2 (cAMP-responsive element binding protein like 2 or CREBL2). This CRE-binding protein-like factor is a small polypeptide of 120 amino acids that contains a basic leucine zipper motif toward the middle of its sequence (amino acids 25 to 74). It binds to L130 with the C-terminal half of the protein (amino acids 59 to 120). The gene for CREBL2 was originally found in a search for genes in a region commonly deleted in hematopoietic malignancies, therefore it has been speculated that CREBL2 may act as a tumor suppressor (Hoornaert et al., 1998). Other CRE-binding proteins appear to bind to proteins important for transcriptional regulation, and these proteins in turn bind to transcription factors as well. The finding that L130 binds to a potential transcription factor provides yet another link between the MAPKAP kinases and transcriptional regulation.

Yeast two-hybrid assays have been performed using the C-NAP1 protein that was previously identified by us to interact with the p38 alpha kinase and was also shown to interact with the Nek2 cell cycle-regulated protein kinase in studies performed by others (Fry et al., 1998). In one such search using C-NAP1 as the protein of interest, the myelin transcription factor MTF1 was shown to be an interactor. MTF1 (also known as MYT1) is a zinc finger-containing nuclear protein that binds to the promoters of proteolipid genes (Yee and Yu, 1998). MTF1 is most highly expressed in the nervous system but is also expressed in low levels in non-neural tissues. The MTF1 protein sequence reveals a few consensus MAP kinase phosphorylation sites, thus raising the possibility that p38 alpha might be capable of interacting with and phosphorylating MTF1 by means of a C-NAP1 bridge.

TNF alpha (tumor necrosis factor) is a protein that initiates the inflammation pathway by binding to the extracellular portion of its receptor and thereby triggering the signal transduction pathway that leads to the activation of the p38 kinases and their downstream kinase substrates (Ledgerwood et al., 1999). It is known that TNF alpha is itself highly regulated, and several factors have been identified that participate in its regulation. Yeast two-hybrid assays have been performed using the TIAR protein which has been implicated in the regulation TNF (tumor necrosis factor) alpha message (Gueydan et al., 1999). Two proteins have been shown to interact with TIAR, profilin II and SEI1. Profilins affect the polymerization of actin in the cytoskeleton (Schluter et al., 1997). At high actin concentrations, profilins prevent actin polymerization whereas under low actin conditions, profilins act to promote polymerization. The significance of the interaction between these two proteins is a bit unclear, however both proteins are known to reside in the cytoplasm. One possibility is that TIAR is tethered to actin filaments by binding to profilin II. TIAR has also been shown to interact with SEI1, a protein first described as a regulator of the cyclin-dependent kinase CDK4 (Sugimoto et al., 1999). SEI1 is rapidly induced in serum-stimulated cells, and it appears to function by antagonizing the activity of the CDK inhibitor p16(INK4a). One interesting possibility is that TIAR itself may be regulated, perhaps by CDK4, via the two proteins' common interaction with SEI1.

The proteins disclosed in the present invention were found to interact with their corresponding proteins in the yeast two-hybrid system. Because of the involvement of the corresponding proteins in the physiological pathways disclosed herein, the proteins disclosed herein also participate in the same physiological pathways. Therefore, the present invention provides a list of uses of these proteins and DNA encoding these proteins for the development of diagnostic and therapeutic tools useful in the physiological pathways. This list includes, but is not limited to, the following examples.

Two-Hybrid System

The principles and methods of the yeast two-hybrid system have been described in detail elsewhere (e.g., Bartel and Fields, 1997; Bartel et al., 1993; Fields and Song, 1989; Chevray and Nathans, 1992). The following is a description of the use of this system to identify proteins that interact with a protein of interest.

The target protein is expressed in yeast as a fusion to the DNA-binding domain of the yeast Ga14p. DNA encoding the target protein or a fragment of this protein is amplified from cDNA by PCR or prepared from an available clone. The resulting DNA fragment is cloned by ligation or recombination into a DNA-binding domain vector (e.g., pGBT9, pGBT.C, pAS2-1) such that an in-frame fusion between the Ga14p and target protein sequences is created.

The target gene construct is introduced, by transformation, into a haploid yeast strain. A library of activation domain fusions (i.e., adult brain cDNA cloned into an activation domain vector) is introduced by transformation into a haploid yeast strain of the opposite mating type. The yeast strain that carries the activation domain constructs contains one or more Ga14p-responsive reporter gene(s), whose expression can be monitored. Examples of some yeast reporter strains include Y190, PJ69, and CBY14a. An aliquot of yeast carrying the target gene construct is combined with an aliquot of yeast carrying the activation domain library. The two yeast strains mate to form diploid yeast and are plated on media that selects for expression of one or more Ga14p-responsive reporter genes. Colonies that arise after incubation are selected for further characterization.

The activation domain plasmid is isolated from each colony obtained in the two-hybrid search. The sequence of the insert in this construct is obtained by the dideoxy nucleotide chain termination method. Sequence information is used to identify the gene/protein encoded by the activation domain insert via analysis of the public nucleotide and protein databases. Interaction of the activation domain fusion with the target protein is confirmed by testing for the specificity of the interaction. The activation domain construct is co-transformed into a yeast reporter strain with either the original target protein construct or a variety of other DNA-binding domain constructs. Expression of the reporter genes in the presence of the target protein but not with other test proteins indicates that the interaction is genuine.

In addition to the yeast two-hybrid system, other genetic methodologies are available for the discovery or detection of protein-protein interactions. For example, a mammalian two-hybrid system is available commercially (Clontech, Inc.) that operates on the same principle as the yeast two-hybrid system. Instead of transforming a yeast reporter strain, plasmids encoding DNA-binding and activation domain fusions are transfected along with an appropriate reporter gene (e.g., lacZ) into a mammalian tissue culture cell line. Because transcription factors such as the Saccharomyces cerevisiae Ga14p are functional in a variety of different eukaryotic cell types, it would be expected that a two-hybrid assay could be performed in virtually any cell line of eukaryotic origin (e.g., insect cells (SF9), fungal cells, worm cells, etc.). Other genetic systems for the detection of protein-protein interactions include the so-called SOS recruitment system (Aronheim et al., 1997). Protein-protein interactions

Protein interactions are detected in various systems including the yeast two-hybrid system, affinity chromatography, co-immunoprecipitation, subcellular fractionation and isolation of large molecular complexes. Each of these methods is well characterized and can be readily performed by one skilled in the art. See, e.g., U.S. Pat. Nos. 5,622,852 and 5,773,218, and PCT published application No. WO 97/27296 and PCT published application No. WO 99/65939, each of which are incorporated herein by reference.

The protein of interest can be produced in eukaryotic or prokaryotic systems. A cDNA encoding the desired protein is introduced in an appropriate expression vector and transfected in a host cell (which could be bacteria, yeast cells, insect cells, or mammalian cells). Purification of the expressed protein is achieved by conventional biochemical and immunochemical methods well known to those skilled in the art. The purified protein is then used for affinity chromatography studies: it is immobilized on a matrix and loaded on a column. Extracts from cultured cells or homogenized tissue samples are then loaded on the column in appropriate buffer, and non-binding proteins are eluted. After extensive washing, binding proteins or protein complexes are eluted using various methods such as a gradient of pH or a gradient of salt concentration. Eluted proteins can then be separated by two-dimensional gel electrophoresis, eluted from the gel, and identified by micro-sequencing. The purified proteins can also be used for affinity chromatography to purify interacting proteins disclosed herein. All of these methods are well known to those skilled in the art.

Similarly, both proteins of the complex of interest (or interacting domains thereof) can be produced in eukaryotic or prokaryotic systems. The proteins (or interacting domains) can be under control of separate promoters or can be produced as a fusion protein. The fusion protein may include a peptide linker between the proteins (or interacting domains) which, in one embodiment, serves to promote the interaction of the proteins (or interacting domains). All of these methods are also well known to those skilled in the art.

Purified proteins of interest, individually or a complex, can also be used to generate antibodies in rabbit, mouse, rat, chicken, goat, sheep, pig, guinea pig, bovine, and horse. The methods used for antibody generation and characterization are well known to those skilled in the art. Monoclonal antibodies are also generated by conventional techniques. Single chain antibodies are further produced by conventional techniques.

DNA molecules encoding proteins of interest can be inserted in the appropriate expression vector and used for transfection of eukaryotic cells such as bacteria, yeast, insect cells, or mammalian cells, following methods well known to those skilled in the art. Transfected cells expressing both proteins of interest are then lysed in appropriate conditions, one of the two proteins is immunoprecipitated using a specific antibody, and analyzed by polyacrylamide gel electrophoresis. The presence of the binding protein (co-immunoprecipitated) is detected by immunoblotting using an antibody directed against the other protein. Co-immunoprecipitation is a method well known to those skilled in the art.

Transfected eukaryotic cells or biological tissue samples can be homogenized and fractionated in appropriate conditions that will separate the different cellular components. Typically, cell lysates are run on sucrose gradients, or other materials that will separate cellular components based on size and density. Subcellular fractions are analyzed for the presence of proteins of interest with appropriate antibodies, using immunoblotting or immunoprecipitation methods. These methods are all well known to those skilled in the art.

Disruption of Protein-protein Interactions

It is conceivable that agents that disrupt protein-protein interactions can be beneficial in many physiological disorders, including, but not-limited to NIDDM, AD and others disclosed herein. Each of the methods described above for the detection of a positive protein-protein interaction can also be used to identify drugs that will disrupt said interaction. As an example, cells transfected with DNAs coding for proteins of interest can be treated with various drugs, and co-immunoprecipitations can be performed. Alternatively, a derivative of the yeast two-hybrid system, called the reverse yeast two-hybrid system (Leanna and Hannink, 1996), can be used, provided that the two proteins interact in the straight yeast two-hybrid system.

Modulation of Protein-protein Interactions

Since the interaction described herein is involved in a physiological pathway, the identification of agents which are capable of modulating the interaction will provide agents which can be used to track the physiological disorder or to use as lead compounds for development of therapeutic agents. An agent may modulate expression of the genes of interacting proteins, thus affecting interaction of the proteins. Alternatively, the agent may modulate the interaction of the proteins. The agent may modulate the interaction of wild-type with wild-type proteins, wild-type with mutant proteins, or mutant with mutant proteins. Agents can be tested using transfected host cells, cell lines, cell models or animals, such as described herein, by techniques well known to those of ordinary skill in the art, such as disclosed in U.S. Pat. Nos. 5,622,852 and 5,773,218, and PCT published application No. WO 97/27296 and PCT published application No. WO 99/65939, each of which are incorporated herein by reference. The modulating effect of the agent can be screened in vivo or in vitro. Exemplary of a method to screen agents is to measure the effect that the agent has on the formation of the protein complex.

Mutation Screening

The proteins disclosed in the present invention interact with one or more proteins known to be involved in a physiological pathway, such as in NIDDM or AD. Mutations in interacting proteins could also be involved in the development of the physiological disorder, such as NIDDM or AD, for example, through a modification of protein-protein interaction, or a modification of enzymatic activity, modification of receptor activity, or through an unknown mechanism. Therefore, mutations can be found by sequencing the genes for the proteins of interest in patients having the physiological disorder, such as insulin, and non-affected controls. A mutation in these genes, especially in that portion of the gene involved in protein interactions in the physiological pathway, can be used as a diagnostic tool and the mechanistic understanding the mutation provides can help develop a therapeutic tool.

Screening for at-risk Individuals

Individuals can be screened to identify those at risk by screening for mutations in the protein disclosed herein and identified as described above. Alternatively, individuals can be screened by analyzing the ability of the proteins of said individual disclosed herein to form natural complexes. Further, individuals can be screened by analyzing the levels of the complexes or individual proteins of the complexes or the mRNA encoding the protein members of the complexes. Techniques to detect the formation of complexes, including those described above, are known to those skilled in the art. Techniques and methods to detect mutations are well known to those skilled in the art. Techniques to detect the level of the complexes, proteins or mRNA are well known to those skilled in the art.

Cellular Models of Physiological Disorders

A number of cellular models of many physiological disorders or diseases have been generated. The presence and the use of these models are familiar to those skilled in the art. As an example, primary cell cultures or established cell lines can be transfected with expression vectors encoding the proteins of interest, either wild-type proteins or mutant proteins. The effect of the proteins disclosed herein on parameters relevant to their particular physiological disorder or disease can be readily measured. Furthermore, these cellular systems can be used to screen drugs that will influence those parameters, and thus be potential therapeutic tools for the particular physiological disorder or disease. Alternatively, instead of transfecting the DNA encoding the protein of interest, the purified protein of interest can be added to the culture medium of the cells under examination, and the relevant parameters measured.

Animal Models

The DNA encoding the protein of interest can be used to create animals that overexpress said protein, with wild-type or mutant sequences (such animals are referred to as “transgenic”), or animals which do not express the native gene but express the gene of a second animal (referred to as “transplacement”), or animals that do not express said protein (referred to as “knock-out”). The knock-out animal may be an animal in which the gene is knocked out at a determined time. The generation of transgenic, transplacement and knock-out animals (normal and conditioned) uses methods well known to those skilled in the art.

In these animals, parameters relevant to the particular physiological disorder can be measured. These parametes may include receptor function, protein secretion in vivo or in vitro, survival rate of cultured cells, concentration of particular protein in tissue homogenates, signal transduction, behavioral analysis, protein synthesis, cell cycle regulation, transport of compounds across cell or nuclear membranes, enzyme activity, oxidative stress, production of pathological products, and the like. The measurements of biochemical and pathological parameters, and of behavioral parameters, where appropriate, are performed using methods well known to those skilled in the art. These transgenic, transplacement and knock-out animals can also be used to screen drugs that may influence the biochemical, pathological, and behavioral parameters relevant to the particular physiological disorder being studied. Cell lines can also be derived from these animals for use as cellular models of the physiological disorder, or in drug screening.

Rational Drug Design

The goal of rational drug design is to produce structural analogs of biologically active polypeptides of interest or of small molecules with which they interact (e.g., agonists, antagonists, inhibitors) in order to fashion drugs which are, for example, more active or stable forms of the polypeptide, or which, e.g., enhance or interfere with the function of a polypeptide in vivo. Several approaches for use in rational drug design include analysis of three-dimensional structure, alanine scans, molecular modeling and use of anti-id antibodies. These techniques are well known to those skilled in the art.

Following identification of a substance which modulates or affects polypeptide activity, the substance may be further investigated. Furthermore, it may be manufactured and/or used in preparation, i.e., manufacture or formulation, or a composition such as a medicament, pharmaceutical composition or drug. These may be administered to individuals.

A substance identified as a modulator of polypeptide function may be peptide or non-peptide in nature. Non-peptide “small molecules” are often preferred for many in vivo pharmaceutical uses. Accordingly, a mimetic or mimic of the substance (particularly if a peptide) may be designed for pharmaceutical use.

The designing of mimetics to a known pharmaceutically active compound is a known approach to the development of pharmaceuticals based on a “lead” compound. This approach might be desirable where the active compound is difficult or expensive to synthesize or where it is unsuitable for a particular method of administration, e.g., pure peptides are unsuitable active agents for oral compositions as they tend to be quickly degraded by proteases in the alimentary canal. Mimetic design, synthesis and testing are generally used to avoid randomly screening large numbers of molecules for a target property.

Once the pharmacophore has been found, its structure is modeled according to its physical properties, e.g., stereochemistry, bonding, size and/or charge, using data from a range of sources, e.g., spectroscopic techniques, x-ray diffraction data and NMR. Computational analysis, similarity mapping (which models the charge and/or volume of a pharmacophore, rather than the bonding between atoms) and other techniques can be used in this modeling process.

A template molecule is then selected, onto which chemical groups that mimic the pharmacophore can be grafted. The template molecule and the chemical groups grafted thereon can be conveniently selected so that the mimetic is easy to synthesize, is likely to be pharmacologically acceptable, and does not degrade in vivo, while retaining the biological activity of the lead compound. Alternatively, where the mimetic is peptide-based, further stability can be achieved by cyclizing the peptide, increasing its rigidity. The mimetic or mimetics found by this approach can then be screened to see whether they have the target property, or to what extent it is exhibited. Further optimization or modification can then be carried out to arrive at one or more final mimetics for in vivo or clinical testing.

Diagnostic Assays

The identification of the interactions disclosed herein enables the development of diagnostic assays and kits, which can be used to determine a predisposition to or the existence of a physiological disorder. In one aspect, one of the proteins of the interaction is used to detect the presence of a “normal” second protein (i.e., normal with respect to its ability to interact with the first protein) in a cell extract or a biological fluid, and further, if desired, to detect the quantitative level of the second protein in the extract or biological fluid. The absence of the “normal” second protein would be indicative of a predisposition or existence of the physiological disorder. In a second aspect, an antibody against the protein complex is used to detect the presence and/or quantitative level of the protein complex. The absence of the protein complex would be indicative of a predisposition or existence of the physiological disorder.

Nucleic Acids and Proteins

A nucleic acid or fragment thereof has substantial identity with another if, when optimally aligned (with appropriate nucleotide insertions or deletions) with the other nucleic acid (or its complementary strand), there is nucleotide sequence identity in at least about 60% of the nucleotide bases, usually at least about 70%, more usually at least about 80%, preferably at least about 90%, and more preferably at least about 95-98% of the nucleotide bases. A protein or fragment thereof has substantial identity with another if, optimally aligned, there is an amino acid sequence identity of at least about 30% identity with an entire naturally-occurring protein or a portion thereof, usually at least about 70% identity, more ususally at least about 80% identity, preferably at least about 90% identity, and more preferably at least about 95% identity.

Identity means the degree of sequence relatedness between two polypeptide or two polynucleotides sequences as determined by the identity of the match between two strings of such sequences, such as the full and complete sequence. Identity can be readily calculated. While there exist a number of methods to measure identity between two polynucleotide or polypeptide sequences, the term “identity” is well known to skilled artisans (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). Methods commonly employed to determine identity between two sequences include, but are not limited to those disclosed in Guide to Huge Computers, Martin J. Bishop, ed., Academic Press, San Diego, 1994, and Carillo, H., and Lipman, D., SIAM J Applied Math. 48: 1073 (1988). Preferred methods to determine identity are designed to give the largest match between the two sequences tested. Such methods are codified in computer programs. Preferred computer program methods to determine identity between two sequences include, but are not limited to, GCG (Genetics Computer Group, Madison Wis.) program package (Devereux, J., et al., Nucleic Acids Research 12(1). 387 (1984)), BLASTP, BLASTN, FASTA (Altschul et al. (1990); Altschul et al. (1997)). The well-known Smith Waterman algorithm may also be used to determine identity.

As an illustration, by a polynucleotide having a nucleotide sequence having at least, for example, 95% “identity” to a reference nucleotide sequence of is intended that the nucleotide sequence of the polynucleotide is identical to the reference sequence except that the polynucleotide sequence may include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These mutations of the reference sequence may occur at the 5 or 3 terminal positions of the reference nucleotide sequence or anywhere between those terminal positions, interspersed either individually among nucleotides in the reference sequence or in one or more contiguous groups within the reference sequence.

Alternatively, substantial homology or (similarity) exists when a nucleic acid or fragment thereof will hybridize to another nucleic acid (or a complementary strand thereof) under selective hybridization conditions, to a strand, or to its complement. Selectivity of hybridization exists when hybridization which is substantially more selective than total lack of specificity occurs. Typically, selective hybridization will occur when there is at least about 55% homology over a stretch of at least about 14 nucleotides, preferably at least about 65%, more preferably at least about 75%, and most preferably at least about 90%. The length of homology comparison, as described, may be over longer stretches, and in certain embodiments will often be over a stretch of at least about nine nucleotides, usually at least about 20 nucleotides, more usually at least about 24 nucleotides, typically at least about 28 nucleotides, more typically at least about 32 nucleotides, and preferably at least about 36 or more nucleotides.

Nucleic acid hybridization will be affected by such conditions as salt concentration, temperature, or organic solvents, in addition to the base composition, length of the complementary strands, and the number of nucleotide base mismatches between the hybridizing nucleic acids, as will be readily appreciated by those skilled in the art. Stringent temperature conditions will generally include temperatures in excess of 30° C., typically in excess of 37° C., and preferably in excess of 45° C. Stringent salt conditions will ordinarily be less than 1000 mM, typically less than 500 mM, and preferably less than 200 mM. However, the combination of parameters is much more important than the measure of any single parameter. The stringency conditions are dependent on the length of the nucleic acid and the base composition of the nucleic acid, and can be determined by techniques well known in the art. See, e.g., Asubel, 1992; Wetmur and Davidson, 1968.

Thus, as herein used, the term “stringent conditions” means hybridization will occur only if there is at least 95% and preferably at least 97% identity between the sequences. Such hybridization techniques are well known to those of skill in the art. Stringent hybridization conditions are as defined above or, alternatively, conditions under overnight incubation at 42° C. in a solution comprising: 50% formamide, 5× SSC (150 mM NaCl, 15 mM trisodium citrate), 50 mM sodium phosphate (pH7.6), 5× Denhardt's solution, 10% dextran sulfate, and 20 μg/ml denatured, sheared salmon sperm DNA, followed by washing the filters in 0.1× SSC at about 65° C.

The terms “isolated”, “substantially pure”, and “substantially homogeneous” are used interchangeably to describe a protein or polypeptide which has been separated from components which accompany it in its natural state. A monomeric protein is substantially pure when at least about 60 to 75% of a sample exhibits a single polypeptide sequence. A substantially pure protein will typically comprise about 60 to 90% W/W of a protein sample, more usually about 95%, and preferably will be over about 99% pure. Protein purity or homogeneity may be indicated by a number of means well known in the art, such as polyacrylamide gel electrophoresis of a protein sample, followed by visualizing a single polypeptide band upon staining the gel. For certain purposes, higher resolution may be provided by using HPLC or other means well known in the art which are utilized for purification.

Large amounts of the nucleic acids of the present invention may be produced by (a) replication in a suitable host or transgenic animals or (b) chemical synthesis using techniques well known in the art. Constructs prepared for introduction into a prokaryotic or eukaryotic host may comprise a replication system recognized by the host, including the intended polynucleotide fragment encoding the desired polypeptide, and will preferably also include transcription and translational initiation regulatory sequences operably linked to the polypeptide encoding segment. Expression vectors may include, for example, an origin of replication or autonomously replicating sequence (ARS) and expression control sequences, a promoter, an enhancer and necessary processing information sites, such as ribosome-binding sites, RNA splice sites, polyadenylation sites, transcriptional terminator sequences, and mRNA stabilizing sequences. Secretion signals may also be included where appropriate which allow the protein to cross and/or lodge in cell membranes, and thus attain its functional topology, or be secreted from the cell. Such vectors may be prepared by means of standard recombinant techniques well known in the art.

### EXAMPLES

The present invention is further detailed in the following Examples, which are offered by way of illustration and are not intended to limit the invention in any manner. Standard techniques well known in the art or the techniques specifically described below are utilized.

### EXAMPLE 1

### Yeast Two-hybrid System

The principles and methods of the yeast two-hybrid systems have been described in detail (Bartel and Fields, 1997). The following is thus a description of the particular procedure that we used, which was applied to all proteins.

The cDNA encoding the bait protein was generated by PCR from brain cDNA. Gene-specific primers were synthesized with appropriate tails added at their 5′ ends to allow recombination into the vector pGBTQ. The tail for the forward primer was 5′-GCAGGAAACAGCTATGACCATACAGTCAGCGGCCGCCACC-3′ (SEQ ID NO: 1) and the tail for the reverse primer was 5′-ACGGCCAGTCGCGTGGAGTGTTATGTCATGCGGCCGCTA-3′ (SEQ ID NO: 2). The tailed PCR product was then introduced by recombination into the yeast expression vector pGBTQ, which is a close derivative of pGBTC (Bartel et al., 1996) in which the polylinker site has been modified to include M13 sequencing sites. The new construct was selected directly in the yeast J693 for its ability to drive tryptophane synthesis (genotype of this strain: Mat α, ade2, his3, leu2, trp1, URA3::GAL1-lacZ LYS2::GAL1-HIS3 ga14del ga180del cyhR2). In these yeast cells, the bait is produced as a C-terminal fusion protein with the DNA binding domain of the transcription factor Ga14 (amino acids 1 to 147). A total human brain (37 year-old male Caucasian) cDNA library cloned into the yeast expression vector pACT2 was purchased from Clontech (human brain MATCHMAKER cDNA, cat. # HL4004AH), transformed into the yeast strain J692 (genotype of this strain: Mat a, ade2, his3, leu2, trp1, URA3::GAL1-lacZ LYS2::GAL1-HIS3 ga14del ga180del cyhR2), and selected for the ability to drive leucine synthesis. In these yeast cells, each cDNA is expressed as a fusion protein with the transcription activation domain of the transcription factor Ga14 (amino acids 768 to 881) and a 9 amino acid hemagglutinin epitope tag. J693 cells (Mat α type) expressing the bait were then mated with J692 cells (Mat a type) expressing proteins from the brain library. The resulting diploid yeast cells expressing proteins interacting with the bait protein were selected for the ability to synthesize tryptophan, leucine, histidine, and β-galactosidase. DNA was prepared from each clone, transformed by electroporation into E. coli strain KC8 (Clontech KC8 electrocompetent cells, cat. # C2023-1), and the cells were selected on ampicillin-containing plates in the absence of either tryptophane (selection for the bait plasmid) or leucine (selection for the brain library plasmid). DNA for both plasmids was prepared and sequenced by di-deoxynucleotide chain termination method. The identity of the bait cDNA insert was confirmed and the cDNA insert from the brain library plasmid was identified using BLAST program against public nucleotides and protein databases. Plasmids from the brain library (preys) were then individually transformed into yeast cells together with a plasmid driving the synthesis of lamin fused to the Ga14 DNA binding domain. Clones that gave a positive signal after β-galactosidase assay were considered false-positives and discarded. Plasmids for the remaining clones were transformed into yeast cells together with plasmid for the original bait. Clones that gave a positive signal after β-galactosidase assay were considered true positives.

### EXAMPLE 2

### Identification of MAPKAP-K2/HSP27 Interaction

A yeast two-hybrid system as described in Example 1 using amino acids 134-325 of MAPKAP-K2 (Swiss Protein (SP) accession no. P49137) as bait was performed. One clone that was identified by this procedure included amino acids 61-206 of HSP27 (SP accession no. P04792).

### EXAMPLES 3-47

### Identification of Protein-Protein Interactions

A yeast two-hybrid system as described in Example 2 using amino acids of the bait as set forth in Table 42 was performed. The clone that was identified by this procedure for each bait is set forth in Table 42 as the prey. The “AA” refers to the amino acids of the bait or prey. The “NUC” refers to the nucleotides of the bait or prey. The Accession numbers refer to GB—GenBank accession numbers or SP—Swiss Protein accession numbers.

TABLE 42

| Ex. | BAIT | ACCESSION | COORDINATES | MUTANT | PREY | ACCESSION | COORDINATES |
|---|---|---|---|---|---|---|---|
| 3 | MAPKAP-K3 | GB: U09578 | NUC 433-1003 | | HIGHLY CHARGED | GB: X59131 | NUC 2832-3411 |
| | | | | | AA SEQ | | |
| 4 | MAPKAP-K3 | GB: U09578 | NUC 433-1003 | | EZF | SP: O43474 | AA 245-473 |
| 5 | MAPKAP-K3 | GB: U09578 | NUC 433-1003 | | K1AA0674 | GB: AB014574 | NUC 2673-3642 |
| 6 | MAPKAP-K3 | GB: U09578 | NUC 433-1003 | | GM88 | GB: AB020662 | NUC 909-1257 |
| 7 | MAPKAP-K3 | GB: U09578 | NUC 433-1003 | | KIAA0216 | GB: D86970 | NUC 3913-4771 |
| 8 | MAPKAP-K3 | GB: U09578 | NUC 433-1003 | | RACK1 | SP: P25388 | AA 195-317 |
| 9 | MAPKAP-K3 | GB: U09578 | NUC 433-1003 | | HRS | GB: U43895 | NUC 1521-2073 |
| 10 | MAPKAP-K3 | GB: U09578 | NUC 433-1003 | | KRML | GB: AF134157 | NUC 781-1045 |
| 11 | MAPKAP-K3 | GB: U09578 | NUC 433-1003 | | TOM1 | GB: AJ006973 | NUC 751-1522 |
| 12 | MAPKAP-K3 | GB: U09578 | NUC 433-1003 | | TPM3 | SP: P12324 | AA 65-199 |
| 13 | MAPKAP-K3 | GB: U09578 | NUC 433-1003 | | ZFM1 | GB: D26120 | NUC 910-1171 |
| 14 | MAPKAP-K3 | GB: U09578 | NUC 433-1003 | | HOMER-3 | GB: AF093265 | NUC 720-1152 |
| 15 | MAPKAP-K3 | GB: U09578 | NUC 433-1003 | | MAX | SP: P25912 | AA 45-149 |
| 16 | MAPKAP-K3 | GB: U09578 | NUC 433-1003 | | ERF-2 | SP: P47974 | AA 7-192 |
| 17 | MAPKAP-K3 | GB: U09578 | NUC 433-1003 | | VIMENTIN | SP: P08670 | AA 309-463 |
| 18 | MAPKAP-K3 | GB: U09578 | NUC 433-1003 | | NUMA1 | GB: Z11583 | NUC 1395-1713 |
| 19 | MAPKAP-K3 | GB: U09578 | NUC 433-1003 | | HSPC161 | GB: AF161510 | NUC 158-1048 |
| 20 | MAPKAP-K3 | GB: U09578 | NUC 433-1003 | | K1AA1026 | GB: AB028949 | NUC 765-1194 |
| 21 | MAPKAP-K3 | GB: U09578 | NUC 742-1003 | | HSP27 | SP: P04792 | AA 73-206 |
| 22 | L130 | SP: P42704 | AA 200-500 | | DYNACTIN | SP: Q14203 | AA 329-451 |
| 23 | L130 | SP: P42704 | AA 1000-1209 | | CREBL2 | GB: AF039081 | NUC 453-636 |
| 24 | PRAK | GB: AF032437 | NUC 201-1104 | | MLK2 | SP: Q02779 | AA 398-556 |
| 25 | PRAK | GB: AF032437 | NUC 201-1104 | K51M | MLK2 | SP: Q02779 | AA 398-556 |
| 26 | PRAK | GB: AF032437 | NUC 201-1104 | K51M | TENASCIN XB | SP: P78530 | AA 616-1098 |
| 27 | PRAK | GB: AF032437 | NUC 201-1104 | K51M | GOLGIN-95 | SP: Q08379 | AA 22-482 |
| 28 | PRAK | GB: AF032437 | NUC 201-1104 | K51M | GOLGIN-95 | SP: Q08379 | AA 76-495 |
| 29 | PRAK | GB: AF032437 | NUC 201-1104 | K51M | GOLGIN-95 | SP: Q08379 | AA 1-145 |
| 30 | PRAK | GB: AF032437 | NUC 201-1104 | T182D | GOLGIN-95 | SP: Q08379 | AA 1-144 |
| 31 | PRAK | GB: AF032437 | NUC 201-1104 | T182D | KENDRIN | SP: O43152 | AA 191-571 |
| 32 | PRAK | GB: AF032437 | NUC 201-1104 | T182D | K1AA0555 | GB: AB011127 | NUC 1617-1983 |
| 33 | PRAK | GB: AF032437 | NUC 201-1104 | K51M, T182D | K1AA0555 | GB: AB011127 | NUC 1617-1983 |
| 34 | PRAK | GB: AF032437 | NUC 201-1104 | K51M, T182D | NUMA1 | GB: Z11583 | NUC 1394-1711 |
| 35 | PRAK | GB: AF032437 | NUC 201-1104 | K51M, T182D | ABP620 | GB: AB029290 | NUC 11355-12486 |
| 36 | PRAK | GB: AF032437 | NUC 201-1104 | K51M, T182D | DYNACTIN | SP: Q14203 | AA 771-999 |
| 37 | PRAK | GB: AF032437 | NUC 201-1104 | K51M, T182D | SMN1 | SP: Q16637 | AA 12-294 |
| 38 | PRAK | GB: AF032437 | NUC 201-1104 | K51M, T182D | HAT1 | SP: O14929 | AA 334-420 |
| 39 | PRAK | GB: AF032437 | NUC 201-1104 | K51M, T182D | HOMER-3 | GB: AF093265 | NUC 774-1152 |
| 40 | PRAK | GB: AF032437 | NUC 201-1104 | K51M, T182D | KINECTIN | GB: Z22551 | NUC 3647-4140 |
| 41 | PRAK | GB: AF032437 | NUC 201-1609 | K51M | BICAUDAL-D | GB: U90028 | NUC 210-1395 |
| 42 | TIAR | SP: Q01085 | AA 1-48 | | PROFILIN II | SP: P35080 | AA 4-91 |
| 43 | TIAR | SP: Q01085 | AA 1-48 | | SEI1 | GB: AF117959 | NUC 131-549 |
| 44 | p38 ALPHA | SP: Q13083 | AA 194-319 | | WBP-2 | GB: U79458 | NUC 103-745 |
| 45 | p38 ALPHA | SP: Q13083 | AA 28-360 | | JNK2 | SP: P45984 | AA 6-40 |
| 46 | p38 GAMMA | SP: P53778 | AA 29-368 | | DLG2 | SP: Q15700 | AA 294-594 |
| 47 | C-NAP1 | GB: AF049105 | NUC 4421-5336 | | MYT1 | SP: Q01538 | AA 504-553 |

### EXAMPLE 48

### Generation of Polyclonal Antibody Against Protein Complexes

As shown above, MAPKAP-K2 interacts with HSP27 to form a complex. A complex of the two proteins is prepared, e.g., by mixing purified preparations of each of the two proteins. If desired, the protein complex can be stabilized by cross-linking the proteins in the complex, by methods known to those of skill in the art. The protein complex is used to immunize rabbits and mice using a procedure similar to that described by Harlow et al. (1988). This procedure has been shown to generate Abs against various other proteins (for example, see Kraemer et al., 1993).

Briefly, purified protein complex is used as immunogen in rabbits. Rabbits are immunized with 100 μg of the protein in complete Freund's adjuvant and boosted twice in three-week intervals, first with 100 μg of immunogen in incomplete Freund's adjuvant, and followed by 100 μg of immunogen in PBS. Antibody-containing serum is collected two weeks thereafter. The antisera is preadsorbed with MAPKAP-K2 and HSP27, such that the remaining antisera comprises antibodies which bind conformational epitopes, i.e., complex-specific epitopes, present on the MAPKAP-K2-HSP27 complex but not on the monomers.

Polyclonal antibodies against each of the complexes set forth in Tables 1-41 are prepared in a similar manner by mixing the specified proteins together, immunizing an animal and isolating antibodies specific for the protein complex, but not for the individual proteins.

### EXAMPLE 49

### Generation of Monoclonal Antibodies Specific for Protein Complexes

Monoclonal antibodies are generated according to the following protocol. Mice are immunized with immunogen comprising MAPKAP-K2/HSP27 complexes conjugated to keyhole limpet hemocyanin using glutaraldehyde or EDC as is well known in the art. The complexes can be prepared as described in Example 48 and may also be stabilized by cross-linking. The immunogen is mixed with an adjuvant. Each mouse receives four injections of 10 to 100 μg of immunogen, and after the fourth injection blood samples are taken from the mice to determine if the serum contains antibody to the immunogen. Serum titer is determined by ELISA or RIA. Mice with sera indicating the presence of antibody to the immunogen are selected for hybridoma production.

Spleens are removed from immune mice and a single-cell suspension is prepared (Harlow et al., 1988). Cell fusions are performed essentially as described by Kohler et al. (1975). Briefly, P3.65.3 myeloma cells (American Type Culture Collection, Rockville, Md.) or NS-1 myeloma cells are fused with immune spleen cells using polyethylene glycol as described by Harlow et al. (1988). Cells are plated at a density of 2×10⁵ cells/well in 96-well tissue culture plates. Individual wells are examined for growth, and the supernatants of wells with growth are tested for the presence of MAPKAP-K2/HSP27 complex-specific antibodies by ELISA or RIA using MAPKAP-K2/HSP27 complex as target protein. Cells in positive wells are expanded and subcloned to establish and confirm monoclonality.

Clones with the desired specificities are expanded and grown as ascites in mice or in a hollow fiber system to produce sufficient quantities of antibodies for characterization and assay development. Antibodies are tested for binding to MAPKAP-K2 alone or to HSP27 alone, to determine which are specific for the MAPKAP-K2/HSP27 complex as opposed to those that bind to the individual proteins.

Monoclonal antibodies against each of the complexes set forth in Tables 1-41 are prepared in a similar manner by mixing the specified proteins together, immunizing an animal, fusing spleen cells with myeloma cells and isolating clones which produce antibodies specific for the protein complex, but not for the individual proteins.

### EXAMPLE 50

### In vitro Identification of Modulators for Protein-protein Interactions

The present invention is useful in screening for agents that modulate the interaction of MAPKAP-K2 and HSP27. The knowledge that MAPKAP-K2 and HSP27 form a complex is useful in designing such assays. Candidate agents are screened by mixing MAPKAP-K2 and HSP27 (a) in the presence of a candidate agent, and (b) in the absence of the candidate agent. The amount of complex formed is measured for each sample. An agent modulates the interaction of MAPKAP-K2 and HSP27 if the amount of complex formed in the presence of the agent is greater than (promoting the interaction), or less than (inhibiting the interaction) the amount of complex formed in the absence of the agent. The amount of complex is measured by a binding assay, which shows the formation of the complex, or by using antibodies immunoreactive to the complex.

Briefly, a binding assay is performed in which immobilized MAPKAP-K2 is used to bind labeled HSP27. The labeled HSP27 is contacted with the immobilized MAPKAP-K2 under aqueous conditions that permit specific binding of the two proteins to form an MAPKAP-K2/HSP27 complex in the absence of an added test agent. Particular aqueous conditions may be selected according to conventional methods. Any reaction condition can be used as long as specific binding of MAPKAP-K2/HSP27 occurs in the control reaction. A parallel binding assay is performed in which the test agent is added to the reaction mixture. The amount of labeled HSP27 bound to the immobilized MAPKAP-K2 is determined for the reactions in the absence or presence of the test agent. If the amount of bound, labeled HSP27 in the presence of the test agent is different than the amount of bound labeled HSP27 in the absence of the test agent, the test agent is a modulator of the interaction of MAPKAP-K2 and HSP27.

Candidate agents for modulating the interaction of each of the protein complexes set forth in Tables 1-41 are screened in vitro in a similar manner.

### EXAMPLE 51

### In vivo Identification of Modulators for Protein-protein Interactions

In addition to the in vitro method described in Example 50, an in vivo assay can also be used to screen for agents which modulate the interaction of MAPKAP-K2 and Hsp27. Briefly, a yeast two-hybrid system is used in which the yeast cells express (1) a first fusion protein comprising MAPKAP-K2 or a fragment thereof and a first transcriptional regulatory protein sequence, e.g., GAL4 activation domain, (2) a second fusion protein comprising HSP27 or a fragment thereof and a second transcriptional regulatory protein sequence, e.g., GAL4 DNA-binding domain, and (3) a reporter gene, e.g., P-galactosidase, which is transcribed when an intermolecular complex comprising the first fusion protein and the second fusion protein is formed. Parallel reactions are performed in the absence of a test agent as the control and in the presence of the test agent. A functional MAPKAP-K2/HSP27 complex is detected by detecting the amount of reporter gene expressed. If the amount of reporter gene expression in the presence of the test agent is different than the amount of reporter gene expression in the absence of the test agent, the test agent is a modulator of the interaction of MAPKAP-K2 and HSP27.

Candidate agents for modulating the interaction of each of the protein complexes set forth in Tables 1-41 are screened in vivo in a similar manner.

While the invention has been disclosed in this patent application by reference to the details of preferred embodiments of the invention, it is to be understood that the disclosure is intended in an illustrative rather than in a limiting sense, as it is contemplated that modifications will readily occur to those skilled in the art, within the spirit of the invention and the scope of the appended claims.

### BIBLIOGRAPHY

Aronheim et al. (1997). Mol. Cell. Biol. 17:3094-3102.

Altschul SF, et al. (1990). J. Mol. Biol. 215: 403-410.

Altschul SF, et al. (1997). NucL. Acids Res. 25:3389-3402.

Asao, H. et al. (1997). J Biol Chem. 272:32785-91.

Ausubel FM, et al. (1992). Current Protocols in Molecular Biology, (J. Wiley and Sons, NY).

Ayer, D. E. et al. (1993). Cell 72:211-22.

Bartel, P. L. et al. (1993). “Using the 2-hybrid system to detect protein-protein interactions.” In: Cellular Interactions in Development: A Practical Approach, Oxford University Press, pp. 153-179.

Bartel, P. L. et al. (1996). Nat Genet 12:72-77.

Bartel, P. L. and Fields, S. (1997). The Yeast Two-Hybrid System. New York: Oxford University Press.

Blackwood, E. M. and Eisenman, R.N. (1991). Science 251:1211-7.

Burch, G. H. et al. (1997). Nat Genet. 17:104-8.

Burk, 0. and Klempnauer, K. H. (1999). Biochim Biophys Acta. 1446:243-52.

Carballo, E. et al. (1998). Science 281:1001-5.

Chen, H. I. et al. (1997). J Biol Chem. 272:17070-7.

Chevray, P. M. and Nathans, D. N. (1992). Proc. Natl. Acad. Sci. USA 89:5789-5793.

Corsini, E. et al. (1999). J Immunol. 163:3468-73.

Diviani, D. et al. (2000). Curr Biol. 10:417-420.

Evans, R. M. (1988). Eur J Cell Biol. 46:152-60.

Fankhauser, C. et al. (1995). Cell 82:435-44.

Fields S and Song O-K (1989). Nature 340:245-246.

Fritzler, M. J. et al. (1993). J Exp Med. 178:49-62.

Fry, A. M. et al. (1998). J Cell Biol. 141:1563-74.

Grammer, A. C. et al. (1998). J Immunol. 161:1183-93.

Gueydan, C. et al. (1999). J Biol Chem. 274:2322-6.

Harlow et al. (1988). Antibodies: A Laboratory Manual (Cold Spring Harbor Laboratory, Cold Spring Harbor, N.Y.).

Hoornaert, I. et al. (1998). Genomics 51:154-7.

Hou, J. et al. (1994). In Vitro Cell Dev Biol Anim. 30A: 111-4.

Karki, S. and Holzbaur, E. L. (1999). Curr Opin Cell Biol. 11:45-53.

Kawasaki, H. et al. (1997). J. Biol Chem. 272:18518-21.

Kim, E. et al. (1996). Neuron 17:103-113.

Kirchner, J. et al. (1999). J. Biol Chem. 380:915-21.

Kohler, G. and Milstein, C. (1975). Nature 256, 495-497.

Kraemer, F. B. et al. (1993). J. Lipid Res. 34, 663-672.

Leanna, C. A. and Hannink, M. (1996). Nucl. Acids Res. 24:3341-3347.

Ledgerwood, E. C. et al. (1999). Lab Invest. 79:1041-50.

Lee, J. C. et al. (1994). Nature 372:739-46.

Lee, J. C. et al. (1996). J Leukoc Biol. 59:152-7.

Lorson, C. L. et al. (1999). Proc Natl Acad Sci USA 96:6307-11.

Lydersen, B. K. and Pettijohn, D. E. (1980). Cell 22:489-99.

Mach, F. et al. (1998). Nature 394:200-3.

Mach, J. M. and Lehmann, R. (1997). Genes Dev. 11:423-35.

Miyazawa, K. et al. (1998). J Biol Chem. 273:24832-8.

Nagata, K. I. et al. (1998). EMBO J. 17:149-58.

Nemoto, S. et al. (1998). Mol Cell Biol. 18:3518-26.

Pirozzi, G. et al. (1997). J Biol Chem. 272:14611-6.

Prasad, G. L. et al. (1993). Proc Natl Acad Sci USA 90:7039-43.

Raingeaud, J. et al. (1995). J Biol Chem. 270:7420-6.

Rogalla, T. et al. (1999). J Biol Chem. 274:18947-56.

Schluter, K. et al. (1997). Biochim Biophys Acta 1359:97-109.

Sheetz, M. P. (1999). Eur J Biochem. 26219-25.

Shields, J. M. et al. (1996). J Biol Chem. 271:20009-17.

Sieweke, M. H. et al. (1996). Cell 85:49-60.

Sluss, H. K. et al. (1994). Mol Cell Biol. 14:8376-84.

Sparks, C. A. et al. (1995). J Cell Sci. 108:3389-96.

Sugimoto, M. et al. (1999). Genes Dev. 13:3027-33.

Takaesu, G. et al. (2000). Mol Cell. 5:649-58.

Tu, J.C. et al. (1998). Neuron 21:717-26.

Varnum, B. C. et al. Mol Cell Biol. 11:1754-8.

Verreault, A. et al. (1998). Curr Biol. 8:96-108.

Wang, Y. et al. (1998). J Biol Chem. 273:2161-8.

Wetmur J G and Davidson N (1968). J Mol. Biol. 31:349-370.

Wu, X. et al. (2000). Curr Opin Cell Biol. 12:42-51. Review.

Yee, K. S. and Yu, V. C. (1998). JBiol Chem. 273:5366-74.

Zhang, D. and Childs, G. (1998). J Biol Chem. 273:6868-77.

Zhang, D. et al. (1998). J Biol Chem. 273:18086-91.

PCT Published Application No. WO 97/27296

PCT Published Application No. WO 99/65939

U.S. Pat. No. 5,622,852

U.S. Pat. No. 5,773,218

What is claimed is:

1. An isolated protein complex comprising two proteins, the protein complex selected from the group consisting of
(a) a complex set forth in Table 1;
(b) a complex set forth in Table 2;
(c) a complex set forth in Table 3;
(d) a complex set forth in Table 4;
(e) a complex set forth in Table 5;
(f) a complex set forth in Table 6;
(g) a complex set forth in Table 7;
(h) a complex set forth in Table 8;
(i) a complex set forth in Table 9;
(h) a complex set forth in Table 10;
(k) a complex set forth in Table 11;
(l) a complex set forth in Table 12;
(m) a complex set forth in Table 13;
(n) a complex set forth in Table 14;
(o) a complex set forth in Table 15;
(p) a complex set forth in Table 16;
(q) a complex set forth in Table 17;
(r) a complex set forth in Table 18;
(s) a complex set forth in Table 19;
(t) a complex set forth in Table 20;
(u) a complex set forth in Table 21;
(v) a complex set forth in Table 22;
(w) a complex set forth in Table 23;
(x) a complex set forth in Table 24;
(y) a complex set forth in Table 25;
(z) a complex set forth in Table 26;
(aa) a complex set forth in Table 27;
(ab) a complex set forth in Table 28;
(ac) a complex set forth in Table 29;
(ad) a complex set forth in Table 30;
(ae) a complex set forth in Table 31;
(af) a complex set forth in Table 32;
(ag) a complex set forth in Table 33;
(ah) a complex set forth in Table 34;
(ai) a complex set forth in Table 35;
(aj) a complex set forth in Table 36;
(ak) a complex set forth in Table 37;
(al) a complex set forth in Table 38;
(am) a complex set forth in Table 39;
(an) a complex set forth in Table 40; and
(ao) a complex set forth in Table 41.

2. The protein complex of claim 1, wherein said protein complex comprises complete proteins.

3. The protein complex of claim 1, wherein said protein complex comprises a fragment of one protein and a complete protein of anther protein.

4. The protein complex of claim 1, wherein said protein complex comprises fragments of proteins.

5. An isolated antibody selectively immunoreactive with the protein complex of claim 1.

6. The antibody of claim 5, wherein said antibody is a monoclonal antibody.

7. A method for diagnosing a physiological disorder in an animal, which comprises assaying for:
(a) whether a protein complex set forth in any one of Tables 1-41 is present in a tissue extract;
(b) the ability of proteins to form a protein complex set forth in any one of Tables 1-41; and
(c) a mutation in a gene encoding a protein of a protein complex set forth in any one of Tables 1-41.

8. The method of claim 7, wherein said animal is a human.

9. The method of claim 7, wherein the diagnosis is for a predisposition to said physiological disorder.

10. The method of claim 7, wherein the diagnosis is for the existence of said physiological disorder.

11. The method of claim 7, wherein said assay comprises a yeast two-hybrid assay.

12. The method o claim 7, wherein said assay comprises measuring in vitro a complex formed by combining the proteins of the protein complex, said proteins isolated from said animal.

13. The method of claim 12, wherein said complex is measured by binding with an antibody specific for said complex.

14. The method of claim 7, wherein said assay comprises mixing an antibody specific for said protein complex with a tissue extract from said animal and measuring the binding of said antibody.

15. A method for determining whether a mutation in a gene encoding one of the proteins of a protein complex set forth in any one of Tables 1-41 is useful for diagnosing a physiological disorder, which comprises assaying for the ability of said protein with said mutation to form a complex with the other protein of said protein complex, wherein an inability to form said complex is indicative of said mutation being useful for diagnosing a physiological disorder.

16. The method of claim 15, wherein said gene is an animal gene.

17. The method of claim 16, wherein said animal is a human.

18. The method of claim 15, wherein the diagnosis is for a predisposition to a physiological disorder.

19. The method of claim 15, wherein the diagnosis is for the existence of a physiological disorder.

20. The method of claim 15, wherein said assay comprises a yeast two-hybrid assay.

21. The method of claim 15, wherein said assay comprises measuring in vitro a complex formed by combining the proteins of the protein complex, said proteins isolated from an animal.

22. The method of claim 21, wherein said animal is a human.

23. The method of claim 21, wherein said complex is measured by binding with an antibody specific for said complex.

24. A method for screening for drug candidates capable of modulating the interaction of the proteins of a protein complex set forth in any one of Tables 1-41, which comprises:
(a) combining the proteins of said protein complex in the presence of a drug to form a first complex;
(b) combining the proteins in the absence of said drug to form a second complex;
(c) measuring the amount of said first complex and said second complex; and
(d) comparing the amount of said first complex with the amount of said second complex,
wherein if the amount of said first complex is greater than, or less than the amount of said second complex, then the drug is a drug candidate for modulating the interaction of the proteins of said protein complex.

25. The method of claim 24, wherein said screening is an in vitro screening.

26. The method of claim 24, wherein said complex is measured by binding with an antibody specific for said protein complexes.

27. The method of claim 24, wherein if the amount of said first complex is greater than the amount of said second complex, then said drug is a drug candidate for promoting the interaction of said proteins.

28. The method of claim 24, wherein if the amount of said first complex is less than the amount of said second complex, then said drug is a drug candidate for inhibiting the interaction of said proteins.

29. A non-human animal model for a physiological disorder wherein the genome of said animal or an ancestor thereof has been modified such that the formation of a protein complex set forth in any one of Tables 1-41 has been altered.

30. The non-human animal model of claim 29, wherein the formation of said protein complex has been altered as a result of:
(a) over-expression of at least one of the proteins of said protein complex;
(b) replacement of a gene for at least one of the proteins of said protein complex with a gene from a second animal and expression of said protein;
(c) expression of a mutant form of at least one of the proteins of said protein complex;
(d) a lack of expression of at least one of the proteins of said protein complex; or
(e) reduced expression of at least one of the proteins of said protein complex.

31. A cell line obtained from the animal model of claim 29.

32. A non-human animal model for a physiological disorder, wherein the biological activity of a protein complex set forth in any one of Tables 1-41 has been altered.

33. The non-human animal model of claim 32, wherein said biological activity has been altered as a result of:
(a) disrupting the formation of said complex; or
(b) disrupting the action of said complex.

34. The non-human animal model of claim 32, wherein the formation of said complex is disrupted by binding an antibody to at least one of the proteins which form said protein complex.

35. The non-human animal model of claim 32, wherein the action of said complex is disrupted by binding an antibody to said complex.

36. The non-human animal model of claim 32, wherein the formation of said complex is disrupted by binding a small molecule to at least one of the proteins which form said protein complex.

37. The non-human animal model of claim 32, wherein the action of said complex is disrupted by binding a small molecule to said complex.

38. A cell in which the genome of cells of said cell line has been modified to produce at least one protein complex set forth in any one of Tables 1-41.

39. A cell line in which the genome of the cells of said cell line has been modified to eliminate at least one protein of a protein complex set forth in any one of Tables 1-41.

40. A method of screening for drug candidates useful in treating a physiological disorder which comprises the steps of:
(a) measuring the activity of a protein selected from the proteins set forth in Tables 1-41 in the presence of a drug,
(b) measuring the activity of said protein in the absence of said drug, and
(c) comparing the activity measured in steps (1) and (2),
wherein if there is a difference in activity, then said drug is a drug candidate for treating said physiological disorder.


SEQUENCE LISTING (2 sequences)

SEQ ID NO: 1 - DNA, 40 bp, Artificial Sequence
  primer for yeast two hybrid
gcaggaaacagctatgaccatacagtcagcggccgccacc

SEQ ID NO: 2 - DNA, 39 bp, Artificial Sequence
  primer for yeast two hybrid
acggccagtcgcgtggagtgttatgtcatgcggccgcta